(19) United States
(12) Patent Application Publication
(10) Pub. No.: US 2014/0087007 A1
(43) Pub. Date: Mar. 27, 2014

(54) BOWEL CLEANSING PREPARATIONS

(71) Applicants: Mark vB. Cleveland, Duxbury, MA (US); Edmund V. Dennett, JR., South Walpole, MA (US); John D. McGowan, Plymouth, MA (US)

(72) Inventors: Mark vB. Cleveland, Duxbury, MA (US); Edmund V. Dennett, JR., South Walpole, MA (US); John D. McGowan, Plymouth, MA (US)

(73) Assignee: Braintree Laboratories, Inc., Braintree, MA (US)

(21) Appl. No.: 13/835,960

(22) Filed: Mar. 15, 2013

Related U.S. Application Data
(60) Provisional application No. 61/706,302, filed on Sep. 27, 2012.

(51) Int. Cl. A61K 33/04; A61K 33/00; A61K 31/765; A61K 33/14

(52) U.S. Cl. CPC A61K 33/04; A61K 33/14; A61K 33/00; A61K 31/765
USPC 424/679; 424/709

(57) ABSTRACT

Disclosed is a method for rapidly cleansing the intestines and colon of a patient by orally administering an aqueous hypertonic sulfate solution, and then an aqueous isotonic solution to induce purgation, the purgations induced resulting in intestinal and colonic cleansing in less than four hours from the administration of the sulfate solution.

### CROSS-REFERENCE TO RELATED APPLICATION

This application claims the benefit of U.S. Provisional Patent application Ser. No. 61/706,302 entitled “Bowel Cleansing Preparations,” which was filed Sep. 27, 2012. The entirety of the aforementioned application is herein incorporated by reference.

### FIELD OF THE INVENTION

This disclosure pertains to internal medicine, gastroenterology, and more specifically, to bowel cleansing.

### BACKGROUND

Colorectal cancer (CRC) is a leading cause of cancer death. The lifetime risk of developing CRC in the US approaches 6%, and almost half of those affected will die of the disease. Despite the usefulness of screening procedures for its detection, CRC is a major cause of morbidity and mortality. In screening procedures for CRC such as sigmoidoscopy, colonoscopy and radiography, it is important that the colon be thoroughly purged and cleansed. In particular, it is essential that as much fecal matter and fluids as possible be removed from the colon to permit adequate visualization of the intestinal mucosa.

Large volume (4 L) orally administered compositions (e.g., NuLYTELY®, TriLyte® containing polyethylene glycol (PEG) in solution) have been developed for use as gastrointestinal washes for diagnostic purposes or for use as cathartic laxatives. Because the solutions are isotonic, patients are required to ingest a significant volume of these solutions, up to one eight ounce glass every ten minutes for a total of one gallon of fluid, to achieve effective purging. However, the large volume required for effective use of this type of lavage formulation is frequently associated with distention, nausea, cramping, vomiting, and significant patient discomfort. Thus, while these formulations are generally effective, they are not well tolerated. Without close supervision, many patients do not take the complete course of preparation.

Small volume (about 300 ml) solutions containing concentrated sodium sulfate and phosphate salts taken in tablespoon size (15 ml) daily amounts have been used as laxatives. An example of this use is (containing sodium sulfate). However, because of their small volumes, when used in this fashion they do not sufficiently clean the colon for diagnostic or surgical procedures.

In an attempt to avoid the problems associated with the high volume types of preparations, ingestible preparations that consist of aqueous solutions of concentrated phosphate salts have been utilized. The aqueous phosphate salt concentrate produces a tremendous osmotic effect on the intra-luminal contents of the bowel, and therefore, evacuation of the bowel occurs with a large influx of water and electrolytes into the colon from the body. These phosphate salt preparations have been developed for the purpose of decreasing the volume required in colonic purgations. One such preparation comprises 480 g per liter monobasic sodium phosphate and 180 g per liter dibasic sodium phosphate in stabilized buffered aqueous solution, and is sold under the brand name Fleet's Phospho-Soda®.

Additionally, non-aqueous tablet or capsule formulations of sodium phosphates and sulfates have been used (U.S. Pat. Nos. 5,997,906, 6,162,464, and 5,616,346). These small volume sulfate/phosphate solutions and non-aqueous formulations have been shown to cause massive electrolyte and fluid shifts that are clinically significant to the patient (US Food and Drug Administration, Center for Drug Evaluation and Research, Sep. 17, 2001; 2002 Physician's Desk Reference, prescribing information for Fleet's Phospho-Soda® and InKine Pharmaceutical's Visicol®).

HalfLytely® and Bisacodyl Tablets Bowel Prep Kit (HalfLytely®) was developed with the goal of reducing the volume of isotonic solution and thereby improving patient compliance. This successful method uses a laxative amount of bisacodyl, which stimulated initial debulking of the colon, prior to the ingestion of 2 L of polyethylene glycol and isotonic electrolyte solution (PEG-ELS). Although volume-related preparation symptoms were greatly reduced with the use of the lower volume HalfLytely® compared to the use of 4 L NuLYTELY®, the bisacodyl component of the preparation has been associated with patient complaints of abdominal cramping, a known adverse event of bisacodyl, as well as rare reports of ischemic colitis.

Thus, what is needed is a reduced-volume preparation designed to be completed over a short duration, while not stimulating abdominal cramping or producing the dangerous adverse side effects associated with sodium phosphate based bowel preparations, and which effectively cleanses the colon.

### SUMMARY

It has been discovered that hypertonic aqueous solutions of sulfates, in combination with isotonic solutions of an osmotic laxative, such as polyethylene glycol (PEG) or PEG, and electrolytes, can effectively cleanse the gastrointestinal tract of a patient in a short period of time (relative to bisacodyl/PEG-based preparations), while not stimulating abdominal cramping or producing the renal injury or clinically significant electrolyte shifts associated with phosphate-based bowel preparation. This discovery has been exploited to produce the methods of the present disclosure

In one aspect, the disclosure provides a method for rapidly cleansing the small and large intestines and colon of a patient. The method comprises; orally administering an effective amount of an aqueous hypertonic sulfate solution to the patient to induce purgation, the solution not producing clinically significant electrolyte shifts, and not containing phosphates; and orally administering an effective amount of an aqueous isotonic solution containing an osmotic laxative to induce purgation. The purgations induced by the solutions administered results in intestinal and colonic cleansing in less than about four hours from the administration of the sulfate solution.

In some embodiments, the aqueous, hypertonic solution comprises Na₂SO₄, K₂SO₄, and MgSO₄. In certain embodiments, the aqueous, hypertonic solution consists essentially of Na₂SO₄, K₂SO₄, and MgSO₄. In some embodiments, the aqueous, hypertonic solution comprises about 8 g to about 26.5 g of Na₂SO₄, about 1.5 g to about 4.7 g of K₂SO₄, and about 1 g to about 2.4 g of MgSO₄. In certain embodiments, the aqueous, hypertonic solution consists essentially of about 8 g to about 26.5 g of Na₂SO₄, about 1.5 g to about 4.7 g of K₂SO₄, and about 1 g to about 2.4 g of MgSO₄. In particular embodiments, the aqueous, hypertonic solution contains about 72.9 mM to about 233 mM sulfate ion. In one embodiment, the aqueous, hypertonic solution comprises about 155 mM sulfate ion.

In some embodiments, the aqueous isotonic solution comprises PEG, and in certain embodiments, the PEG is PEG 3350. In some embodiments, the aqueous, isotonic solution comprises from about 105 g to about 315 g PEG. In one embodiment, the aqueous, isotonic solution comprises about 118 g PEG, and in another, the aqueous isotonic solution comprises about 210 g PEG.

In certain embodiments, the aqueous, isotonic solution further comprises Na₂SO₄, Na₂CO₃, NaCl, and KCl. In some embodiments, the aqueous, isotonic solution further comprises about 5.0 g to about 18.0 g Na₂SO₄, about 1.7 g to about 5.5 g Na₂CO₃, about 1.5 g to about 4.4 g NaCl, and about 0.75 g to about 2.3 g KCl. In a particular embodiment, the aqueous, isotonic solution further comprises about 11.37 g Na₂SO₄, about 3.37 g Na₂CO₃, about 2.93 g NaCl, and about 1.5 g KCl.

In other embodiments, the aqueous, isotonic solution further comprises Na₂CO₃, NaCl, and KCl. In some embodiments, the aqueous, isotonic solution further comprises about 1.4 g to about 4.3 g Na₂CO₃, about 2.8 g to about 8.4 g NaCl, and about 0.4 g to about 1.1 g KCl. In a particular embodiment, the aqueous, isotonic solution further comprises about 2.8 g Na₂CO₃, about 5.6 g NaCl, and about 0.74 g KCl.

In yet other embodiments the aqueous, isotonic solution further comprises ascorbic acid, sodium ascorbate, Na₂SO₄, NaCl, and KCl. In certain embodiments, the osmotic laxative comprises about 2.0 g to about 5.0 g ascorbic acid, about 2.0 g to about 7.0 g sodium ascorbate, about 3.5 g to about 8.0 g Na₂SO₄, about 1.2 g to about 3.0 g NaCl, and about 0.25 g to about 1.5 g KCl. In some embodiments, the isotonic solution comprises about 2.35 g to about 4.7 g ascorbic acid, and about 2.95 g to about 5.9 g sodium ascorbate, about 3.75 g to about 7.5 g Na₂SO₄, about 1.345 g to about 2.691 g NaCl, and about 0.507 g to about 1.015 g KCl.

In some embodiments, about 100 ml to about 900 ml of the aqueous, hypertonic sulfate solution is administered, and in other embodiments, about 100 ml to about 500 ml of the aqueous, hypertonic sulfate solution is administered. In a particular embodiment, about 330 ml or about 480 ml of the aqueous, hypertonic sulfate solution is administered.

In some embodiments about 1 L to about 3 L of the aqueous isotonic solution is administered. In particular embodiments, about 2 L of the aqueous isotonic solution is administered.

In certain embodiments the aqueous, isotonic solution is administered immediately after ingestion of the aqueous, hypertonic sulfate solution. In other embodiments, the aqueous, isotonic solution is administered about 0.5 hour to about 2 hours after ingestion of the aqueous, hypertonic sulfate solution. In some embodiments, the aqueous, isotonic solution is administered about 1 hour to about 2 hours after ingestion of the aqueous, hypertonic sulfate solution.

In other embodiments, water is orally administered immediately after or within 2 hours after the step of orally administering the aqueous, hypertonic sulfate solution. In other embodiments, the water is administered after the isotonic solution is administered.

In another aspect, the disclosure provides a method for rapidly cleansing the small and large intestines and colon of a patient. The method comprises: orally administering about 100 ml to about 900 ml of an aqueous hypertonic solution consisting essentially of about 17.5 g Na₂SO₄, about 1.6 g MgSO₄, and about 3.1 g K₂SO₄ to induce purgation, the solution not producing any clinically significant electrolyte shifts, and not containing phosphates; orally administering water; and then orally administering from about 1 L to about 3 L of an aqueous, isotonic solution comprising about 168 g PEG, about 11.37 g Na₂SO₄, about 3.37 g Na₂CO₃, about 2.93 g NaCl, and about 1.985 g KCl to induce purgation. The purgations induced by the solutions administered result in intestinal and colonic cleansing in less than about four hours from the administration of the sulfate solution.

In another aspect, the disclosure provides a method for rapidly cleansing the small and large intestines and colon of a patient. The method comprises: orally administering about 100 ml to about 900 ml of an aqueous hypertonic solution consisting essentially of about 17.5 g Na₂SO₄, about 1.6 g MgSO₄, and about 3.1 g K₂SO₄ to induce purgation, the solution not producing any clinically significant electrolyte shifts, and not containing phosphates; orally administering water; and then orally administering from about 1 L to about 3 L of an aqueous, isotonic solution comprising about 210 g PEG, about 2.86 g Na₂CO₃, about 5.6 g NaCl, and about 0.74 g KCl to induce purgation. The purgations induced by the solutions administered result in intestinal and colonic cleansing in less than about four hours from administration of the sulfate solution.

### DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

### DEFINITIONS

Unless otherwise defined, all technical and scientific terms used herein have the same meaning as commonly understood by one of ordinary skill in the art to which this invention belongs.

For convenience, certain terms employed in the specification, examples, and appended claims are collected here. Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean a value − or +20% of a given numerical value. Thus, about 60% means a value of between 60%−20% of 60 and 60%+20% of 60 (i.e., between 48% and 72%).

The term “composition” refers to a sulfate-containing pharmaceutical material or product which can be in the form of a solution, suspension, tablet, capsule, or powder.

The term “aqueous hypertonic solution” is used herein to refer to a water-based mixture of poorly absorbable sulfate salts that creates an osmotic pressure gradient between the bowel and bodily fluids large enough to induce the movement of water from the body into the bowel, thereby producing a purgation.

The term “isotonic solution” is used herein to refer to a solution that has the same salt concentration or the same effective osmotic concentration as found in cells in the body.

The terms “cleansing” and “colonic cleansing” are used herein to refer to removal of stool material from the bowel such that the bowel can be effectively examined, e.g., by colonoscopy or sigmoidoscopy. One nonlimiting step performed to achieve at least partial cleansing is colonic purgation, and more than one colonic purgation resulting in colonic cleansing.

The term “purgation” is used herein to refer to an evacuation of a copious amount of stool from the bowels after administration of a laxative.

The term “clinically-significant” is used herein to refer to electrolyte and fluid shifts which are massive movements of electrolytes and fluids causing alterations in blood chemistry that are outside the normal upper or lower limits of their normal range, and which cause adverse clinical effects (such as renal and/or cardiac disturbances and disturbances caused by sodium phosphate) as determined by a medical professional.

The term “abnormal values” is used herein as values that are outside the upper or lower limits of a determined normal range.

The phrase “effective amount” as used herein means that amount of one or more agent, material, or composition comprising one or more agents according to the present disclosure that is effective for producing some desired effect in an animal. More particularly, in the present disclosure, an “effective amount” is that amount and combination of salts necessary to produce a colonic purgation while not producing clinically significant electrolyte shifts. In general, it is recognized that when an agent is being used to achieve a therapeutic effect, the actual amount which comprises the “effective amount” will vary depending on a number of conditions including, but not limited to, the physical form of the composition being administered (such as a solution or tablet), the particular condition being treated, the severity of the disease, the size and health of the patient, and the route of administration. A skilled medical practitioner can readily determine the appropriate amount using methods well known in the medical arts (e.g., see, e.g., Remington: The Science and Practice of Pharmacy (20^{th} ed) Limmer, Editor, Lipincott, Williams, & Wilkins (2000)).

In one nonlimiting example, an “effective amount” pertains to an amount of a sulfate solution which, after administration or ingestion, at least induces purgation of the GI tract of its contents. In another nonlimiting example, an “effective amount” pertains to an amount of an aqueous isotonic solution which, after administration to a patient, induces purgation, and which, along with the sulfate solution, can ultimately result in cleansing the GI tract. The “effective amount” may be administered at one time or at multiple times.

The phrase “pharmaceutically acceptable” is employed herein to refer to those compounds, materials, compositions, and/or dosage forms which are, within the scope of sound medical judgment, suitable for use in contact with the tissues of human beings, animals and plants without excessive toxicity, irritation, allergic response, or other problem or complication, commensurate with a reasonable benefit/risk ratio.

A “patient” is a mammal, such as a human, cow, dog, cat, horse, rat, rabbit, etc.

The terms “administrating” and “ingesting” are used interchangeably to refer to the oral provision of something to the gastrointestinal tract of a patient.

It has been discovered that hypertonic aqueous solutions of sulfates, in combination with an isotonic solution of an osmotic laxative, such as polyethylene glycol (PEG) or PEG and electrolytes, can effectively cleanse the colon in a short period of time (relative to bisacodyl/PEG-based preparations), while not stimulating abdominal cramping or producing the renal injury or clinically significant electrolyte shifts associated with phosphate-based bowel preparation.

The aqueous, hypertonic sulfate solution does not contain phosphates but contains inorganic sulfate in total amounts ranging from 125 mM to 250 mM, such as about 154 mM. The sulfate can be in the form of various sulfate salts, which, when in solution and in combination, are in ionic form and are effective to produce colonic purgation while maintaining an electrolyte balance. For example, useful sulfate salts include Na₂SO₄, MgSO₄, and K₂SO₄. The amounts of the sulfate salts may differ in order to provide electrolyte balance within the body. For example, Na₂SO₄ may be present from about 0.01 g to about 40.0 g, from about 0.1 g to about 20.0 g, from about 1.0 g to 10.0 g, or from about 8 g to about 26.5 g. Administered amounts of MgSO₄ may be from about 0.01 g to about 40.0 g, from about 0.1 g to about 20.0 g, from about 1.0 g to about 10.0 g, or from about 1.5 g to about 4.7 g. Administered amounts of K₂SO₄ may be from about 0.01 g to about 20.0 g, from about 0.1 g to about 10.0 g, from about 0.5 g to about 5.0 g, or from about 1.0 g to about 2.4 g.

For administration, the sulfate salts are dissolved in a volume of water to produce a hypertonic aqueous solution. A volume of less than 1 L of water is well tolerated by most patients, and volumes of less than 500 ml, such as about 100 ml and about 500 ml, about 200 ml to about 450 ml, and from about 300 ml to about 400 ml are useful. For example, a volume of 330 ml is useful as an oral form.

Following ingestion of the aqueous, hypertonic sulfate solution, an aqueous isotonic, electrolyte-balanced aqueous solution of an osmotic laxative agent is administered and orally ingested. This isotonic solution is administered immediately, up to about 10 minutes, up to about 20 minutes, up to about 30 minutes, up to about 40 minutes, up to about 50 minutes, up to about 60 minutes, up to about 70 minutes, up to about 80 minutes, up to about 90 minutes, up to about 100 minutes, up to about 110 minutes, or up to about two hours after the hypertonic sulfate solution is administered.

One useful osmotic laxative is PEG, which ranges in molecular weight from about 3000 D to about 8000 D. For example, PEG 3350 is a useful osmotic laxative. In some embodiments, from about 100 g to about 300 g, from about 105 g to about 315 g, from about 150 g to about 300 g, from about 200 g to about 350 g, or from about 105 g to about 315 g of PEG are administered in an aqueous, isotonic solution. In some nonlimiting examples, about 118 g of PEG 3350 are used, and in others, about 210 g of PEG are used.

The PEG is dissolved in an aqueous solution having a volume of from 0.5 L to about 3 L. In some examples, the PEG administered is in a volume of from about 1 L to about 3 L, from about 1.2 L to about 2.9 L, from about 1.3 L to about 2.8 L, from about 1.4 L to about 2.7 L, from about 1.5 L to about 2.6 L, from about 1.5 L to about 3 L, or from about 1.5 L to about 2.5 L. In one example, the PEG is dissolved in 2 L of an aqueous solution.

The PEG solution may contain electrolytes to maintain electrolyte balance within the body. Electrolytes, such as NaCl, KCl, Na₂CO₃, and/or Na₂SO₄ in such concentrations that are found within normal cells in the body, are used. In one non-limiting example, 2.86 g Na₂CO₃, about 5.6 g NaCl, and about 0.74 g KCl are used. Sodium sulfate, sodium ascorbate, and/or ascorbate acid are also useful in combination with at least some of these salts.

Useful amounts of electrolyte salts include from 1.5 g to 4.4 g NaCl, from 0.75 g to 2.3 g KCl, from 1.7 g to 5.5 g Na₂CO₃, from 5.0 g to 18.0 g Na₂SO₄, from 2.3 g to 4.7 g ascorbic acid, and/or from 2.7 g to 6.0 g sodium ascorbate.

For example, electrolyte-containing osmotic PEG solutions such as NuLYTELY® (PEG, NaCl, KCl, Na₂CO₃, Na₂SO₄) (Braintree Laboratories, Inc., Braintree, Mass.) or GoLYTELY® (PEG, NaCl, KCl, Na₂CO₃) (Braintree Laboratories, Inc., Braintree, Mass.) and MoviPrep® (PEG, Na₂SO₄, NaCl, KCl, ascorbic acid, sodium ascorbate) (Salix Pharmaceuticals, Inc., Raleigh, N.C.) are useful. To maintain electrolyte balance, changes in the salt concentrations may accompany changes in the PEG concentration.

Water may be orally administered in some cases directly or from 0-2 hours after administration of the sulfate solution. In addition or alternatively, water may be orally administered after the isotonic solution.

The sulfate salt solutions such as those described herein were investigated for their efficacy as colon debulking agents because bisacodyl has been associated with reports of abdominal cramping. Four different solutions were prepared with varying amounts of sodium, magnesium, and potassium sulfate salts and each containing a total sulfate amount of 125 mM. The sulfate salt composition of each solution is shown in Table 1.

TABLE 1
Composition of Sulfate Formulations

| mMole | Soln 1 | Soln 2 | Soln 3 | Soln 4 |
|---|---|---|---|---|
| Na₂SO₄ | 142.5 | 114 | 128 | 128 |
| MgSO₄ | 0 | 0 | 0 | 8.3 |
| K₂SO₄ | 20 | 16 | 18 | 18 |
| Volume (ml) | 165 | 165 | 165 | 100 |

To demonstrate that these sulfate solutions are effective for colonic purgation without producing clinically significant electrolyte and fluid shifts, 27 normal volunteers were given one amount of a laxative which was either one of four different experimental sulfate solutions or another known laxative, or bowel cleansers. These included bisacodyl (20 mg), a PEG and balanced electrolyte isotonic preparation (NuLYTELY®, (2 L or 4 L) or HalfLytely®+Bisacodyl Bowel Prep Kit, (2 L PEG and electrolytes and 10 mg bisacodyl). Bowel movements and urine were collected for 24 hours. The stool output of the subjects were compared and evaluated for movement of electrolytes between the solution and the subject.

The results measured were mean stool volume output and are shown below in Table 2 using non-sulfate laxatives and in Table 3 using sulfate laxatives.

TABLE 2
Mean Stool Volume Output Using Non-Sulfate Laxatives

| | Bisacodyl | 2 L | 4 L | |
|---|---|---|---|---|
| | 20 mg | NuLYTELY ® | NuLYTELY ® | HalfLytely ® |
| N | 11 | 6 | 4 | 7¹ |
| Output | 757 | 1659 | 3861 | 2403 |
| (g) | | | | |
| (SD) | (260) | (231) | (168) | (577) |
| % Solids | 50.4 | 15.0 | 4.8 | 2.6 |
| (SD) | (18.7) | (11.2) | (4.5) | (2.2) |

¹One patient in the HalfLytely ® group did not have their percent solids measured so sample size was 6.

TABLE 3
Mean Stool Volume Output Using Sulfate Formulations

| | | | | | Soln. 4 + |
|---|---|---|---|---|---|
| | Soln. 1 | Soln. 2 | Soln. 3 | Soln. 4 | PEG |
| n | 1 | 1 | 3 | 5 | 1 |
| Output (g) | 1536 | 1080 | 1082 | 1308 | 2298 |
| (SD) | | | (215) | (281) | |
| % Solids | 3.6 | 10.7 | 14.1 | 12.0 | 1.4 |
| (SD) | | | (4.0) | (2.1) | |

As shown in Tables 2 and 3, the sulfate formulations produced greater stool output and lower percent stools solids than 20 mg of bisacodyl, indicating that sulfate is an effective debulking agent if ingested prior to the administration of an isotonic solution of PEG. In addition, combining sulfate Soln. 4 with 2 L NuLYTELY® yields results equivalent to the approved bowel preparation, HalfLytely®. This was confirmed by the results obtained when Soln. 4 was administered followed by 2 L of another PEG and electrolyte solution (NuLYTELY®).

To determine if the present method caused significant electrolyte changes, blood samples were taken from participants about 2 hours prior to ingestion of the laxative and then 2 hours after ingestion. Various analytes in serum were measured and were in the normal range. The sulfate formulation had little or no effect on any of the analytes measured, including serum osmolality and serum electrolytes.

The sulfate formulation was further refined in another study (Patel, et al. (2009) Am. J. Gastroenterol. 104(4):953-65) which showed that the sulfate formulation is equivalent to the marketed bowel preparations NuLYTELY® and EZPrep®.

The safety and efficacy of the sulfate/PEG method was then further compared to the FDA-approved Bisacodyl/PEG (HalfLytely®) method in a clinical study, as described in the examples below. The use of the present sulfate/PEG method resulted in a significantly shorter preparation time than use of the Bisacodyl/PEG method (averaging 3.7 hours compared to 5.5 hours) with no statistically significant differences between the methods with respect to treatment-emergent adverse effects. Finally, the present sulfate/PEG method achieved significantly greater “Excellent” preps and a better average cleansing than the Bisacodyl/PEG method. Accordingly, the present method is a highly improved alternative to existing bowel cleansing methods.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Sulfate/PEG vs. Bisacodyl/PEG Treatment

### A. Formulations Tested

The “sulfate salt/PEG” preparation included a 16 ounce (330 ml) aqueous, hypertonic sulfate solution containing 125 mM sulfate, total (17.510 g Na₂SO₄, 1.6 g MgSO₄ (anhydrous), and 3.13 g K₂SO₄) diluted up to 16 ounces (170.41 g) of purified water. Flavoring and preservatives known in the art can optionally be added as well. This solution can be made from a concentrated 6 ounce solution, and diluted up to 16 ounces with purified water.

The 2 L aqueous, isotonic PEG solution contains 210 g PEG 3350, 5.6 g NaCl, 2.86 g NaCo3, and 0.74 g.

The “bisacodyl/PEG” preparation (HalfLytely®) included two 5 mg bisacodyl tablets and a 2 L solution of HalfLytely® containing 210 g PEG 3350, 2.86 g of sodium bicarbonate, 5.6 g of sodium chloride, 0.74 g of potassium chloride, and 1 g of flavoring ingredient (optional). The solution contains 31.3 mMole/L PEG, 65 mMole/L sodium, 53 mMole/L chloride, 17 mMole/L bicarbonate, and 5 mMole/L potassium.

### B. Administration

In this single blind, active controlled study, the sulfate salt/PEG preparation or the bisacodyl/PEG preparation (HalfLytely®) kits were provided to 366 male and female outpatients that were at least 18 years of age and otherwise in good health requiring colonoscopy for routinely accepted indications. The order of preparation assignment was determined according to a computer generated randomization schedule. Patients self-administered the assigned study preparation in two amounts on the day before their scheduled colonoscopy.

Study patients were provided with a treatment questionnaire to record food consumption, any vomiting episodes and the date and time of preparation. Prior to the colonoscopy, study patients also completed a symptom questionnaire to report their overall experience with the preparation. Blood samples were collected at baseline and pre-colonoscopy for chemistry and hematology analysis.

The sulfate/PEG preparation was supplied as a kit containing one 6 ounce bottle of sulfate solution and one 2 L bottle of PEG-ELS. The bisacodyl/PEG preparation was supplied as a kit containing two 5 mg bisacodyl tablets and one 2 L bottle of PEG-ELS. The HalfLytely® and Bisacodyl Tablets Bowel Prep Kit (Braintree Laboratories, Inc.) was used as the control preparation.

All patients (both treatments) were instructed to consume clear liquids only on the day prior to colonoscopy. This clear liquid diet continued until after completion of the colonoscopy. For the sulfate salt/PEG group, starting at about 6:00 P.M. on the evening before colonoscopy, patients were instructed to pour the contents of the 6 ounce bottle of study preparation into the provided mixing cup and to fill the cup with water to the fill line (16 oz) and then drink the entire cup of solution. Patients were further instructed to drink one additional 16 ounce glass of water over the next two hours. Two hours after starting the first amount (approximately 8:00 P.M.), patients were instructed to begin drinking the 2 L of PEG-ELS solution at a rate of one 16 ounce glass every 20 minutes until the jug was empty. Patients were recommended to drink at least one additional 16 ounce glass of water on the evening prior to colonoscopy.

For the bisacodyl/PEG group, between approximately 12:00 P.M. and 3:00 P.M. on the day prior to colonoscopy, patients were instructed to take the two 5 mg bisacodyl tablets with water. After waiting for a bowel movement (or maximum of 6 hours after taking the bisacodyl tablets) patients were instructed to drink the 2 L HalfLytely® solution at a rate of 8 ounces every 10 minutes.

### C. Measurements

Blinded study investigators rated the quality of each colonoscopy according to a four point scale ranging from “poor” to “excellent.” This scale is shown below in Table 4.

TABLE 4
Colonoscopist Colon Cleansing Scores

| Score | Grade | Description |
|---|---|---|
| 1 | Poor | Large amounts of fecal residue, |
| | | additional cleansing required |
| 2 | Fair | Enough feces or fluid to prevent a |
| | | completely reliable exam |
| 3 | Good | Small amounts of feces or fluid not |
| | | interfering with exam |
| 4 | Excellent | No more than small bits of adherent |
| | | feces/fluid |

For the primary efficacy analysis, grades 3 and 4 were considered “successful” and grades 1 and 2 were considered “failures.” Each examination was also rated as to whether or not cleansing was adequate for examination and the need for re-preparation.

Safety assessments included adverse event monitoring as well as pre and post physical examination. Blood samples were collected at each study visit for chemistry and hematology analysis.

Patients completed a symptom questionnaire which asked them to provide an overall rating of their preparation related symptoms of stomach cramping, stomach bloating, nausea and overall discomfort.

### D. Statistical Methods

Statistical Analysis Plan: The primary efficacy analysis was based upon a modified intent-to-treat (ITT) analysis and included all patients randomized that took any portion of study preparation. Patients that did not undergo colonoscopy because of inadequate preparation or preparation related adverse events were considered failures for the primary efficacy endpoint. However, patients that took study preparation but withdrew prior to colonoscopy for reasons unrelated to safety or efficacy were excluded from efficacy analyses. Patients that underwent colonoscopy had a determination of cleansing success based on the colonoscopists' score of cleansing (Table 4).

Success rate was analyzed using CMH Chi-square adjusting for the effect of investigator site. The formal hypothesis test result (p-value) for treatment difference is presented together with a two-sided 95% confidence interval for the difference. A lower CI bound greater than −15% will establish non-inferiority between BLI850 and HalfLytely® for a non-inferiority margin of 15%.

In addition, results are also presented using a non-inferiority test based upon the difference D=P 1−P2.
Null Hypothesis H0: P1−P2<=D0 versus Alternative Hypothesis H1: P1−P2=D1>D0, where P1 is the BLI850 group (treatment group) and P2 is the HalfLytely® group (reference or control group) and D0 is the acceptable margin of equivalence equal to an absolute margin of 15%.

Results of laboratory tests for the change from screening and group differences were tested using ANOVA. In addition, shift tables are presented to describe changes in lab parameter values between baseline and post-treatment time points using normal range categories (low, normal, high). A second analysis was performed by age subgroup (<65, ≧65, ≧75 years of age).

Symptom questionnaire data for patient reported individual symptoms for Overall Experience (nausea, stomach cramping, stomach bloating and overall discomfort) were tested using ANOVA with terms for treatment, site, and their interaction.

### E. Results

362 patients of the 366 patients that received their study preparation fully completed the study (defined as patients that had a colonoscopy). The primary efficacy analysis was based on 364 patients. All patients enrolled that took the study preparation and underwent colonoscopy (n=362) were included. Two patients were also included in the primary efficacy responder analyses as failures because they could not undergo colonoscopy due to a concurrent adverse event or non-compliance with preparation administration.

Patients reported to their study center for scheduled colonoscopy after completing their bowel preparation and returned study drug materials were reviewed for treatment compliance. A compliant patient was defined as one who returned no more than 4 ounces of the PEG-ELS solution, or who reported taking all solution. More HalfLytely® patients completed the entire preparation than BLI850 patients (94% to 87%, respectively, p=0.033), although compliance for both preparations was considered excellent.

Patient compliance with the study preparation administration was excellent, averaging more than 87%. Preparation time was significantly shorter for BLI850 patients, averaging 3.69 hours compared to 5.52 hours for HalfLytely® (p<0.001).

Table 5 includes the colonoscopy cleansing scores of all 364 patients that had a colonoscopy or withdrew due to safety or non-compliance. Sulfate salt/PEG achieved significantly more “Excellent” preparations than Bisacodyl/PEG (p=0.010). In addition, the average cleansing score was also significantly higher for sulfate salt/PEG.

TABLE 5
Preparation Cleansing Score

| | Sulfate/PEG | Bisacodyl/PEG | |
|---|---|---|---|
| Score | n (%) | n (%) | P^{1,2} |
| 4 | 84 | 67 | 0.010 |
| Excellent | (47.7%) | (35.6%) | |
| 3 | 74 | 90 | |
| Good | (42.0%) | (47.9%) | |
| 2 | 13 | 25 | |
| Fair | (7.4%) | (13.3%) | |
| 1 | 4 | 5 | |
| Poor | (2.3%) | (2.7%) | |
| Missing | 1 | 1 | |
| | (0.6%) | (0.5%) | |
| Mean Score³ | 3.36 | 3.17 | 0.016 |

¹P-value comparing excellent preps is from a CMH, controlling for site;
²P-value for mean score is from a one-way ANOVA with term for reatment.
³05040 and 10003 were excluded as non-evaluable for efficacy analysis. 05006 and 06027 are included as “missing”

Sulfate salt/PEG patients had a higher percentage of successful preparations compared to bisacodyl/PEG patients (90% to 84%, respectively). Non-inferiority testing revealed a highly statistically significant result (p<0.001), supporting the hypothesis that same day preparation with sulfate salt/PEG is non-inferior to bisacodyl/PEG. Sulfate salt/PEG can be considered equivalent with respect to cleansing efficacy to the FDA approved bisacodyl/PEG control.

A sensitivity analysis of the primary endpoint using a true ITT population (all randomized subjects) confirms the previous conclusion that sulfate salt/PEG is non-inferior to bisacodyl/PEG, with 80.6% of sulfate salt/PEG ITT patients having a successful preparation compared to 79.3% of bisacodyl/PEG patients (p<0.001).

### F. Adverse Side Effects

Patients rated their symptoms of cramping, stomach bloating, nausea and overall discomfort using a five point scale where a score of 1=“None,” 2=“Mild,” 3=“Bothersome,” 4=“Distressing,” and a score of 5=“Severely distressing.” Patients were instructed to rate these symptoms using the symptom questionnaire at Visit 2. Symptom data at their final visit are shown below in Table 6.

TABLE 6
Mean Patient Symptom Ratings at Final Visit

| | Sulfate/PEG | Bisacodyl/PEG | |
|---|---|---|---|
| Symptom¹ | (n = 176) | (n = 190) | P² |
| Cramping (SD) | 1.50 (0.70) | 1.55 (0.77) | 0.393 |
| Stomach Bloating (SD) | 1.74 (0.87) | 1.62 (0.82) | 0.177 |
| Nausea (SD) | 1.70 (1.02) | 1.65 (0.97) | 0.818 |
| Overall (SD) | 2.06 (0.98) | 1.76 (0.80) | 0.032 |

¹1 = None; 2 = Mild; 3 = Bothersome; 4 = Distressing; 5 = Severely Distressing
²p-value for difference between treatments by ANOVA

Patients generally had good experiences with both preparations as indicated by the low average symptom scores (most ranged between None (1) and Mild (2)). Although there was no difference between the preparations for symptoms of cramping, bloating and nausea, a very small but statistically significant difference in the general category “overall discomfort” (p=0.032) favoring Bisacodyl/PEG was observed. The difference represented +0.3 or less than one third of a unit score compared to Bisacodyl//PEG in the range between “None” to “Mild” and is considered clinically insignificant. The overall discomfort scores reported in this study are within the narrow range seen in previous studies of approved preparations.

### G. Summary

Patients in the sulfate salt/PEG group had better cleansing, suitable for colonoscopy, relative to bisacodyl/PEG patients (90% vs. 84%, respectively). Furthermore, sulfate salt/PEG patients experienced significantly more “Excellent” preparations than bisacodyl/PEG (48% vs. 36%, respectively, p=0.010). Results from this study support the conclusion that same day preparation with sulfate salt/PEG is equivalent to same day preparation with bisacodyl/PEG with respect to preparation efficacy, and results in a greater number of “Excellent” preparations. Notably, the time to complete the method with successful results was shorter (about 3.69 hours) than to complete the Bisacodyl/PEG method.

### Example 2

### Clinical Evaluations

Blood samples of the patients taking part in the testing described above in Example 1 were analyzed at baseline and after preparation (just prior to colonoscopy). Table 7 shows means for each study visit as well as the change from baseline.

TABLE 7
Mean (SD) Chemistry Values by Visit
ITT Population

| Analyte | Normal | | | | | |
|---|---|---|---|---|---|---|
| (units) | Range¹ | Treatment | Baseline | Visit 2 | Δ to Visit 2 | p² |
| Albumin | 3.7-4.9 | BLI-850* | 4.48 (0.26) | 4.51 (0.27) | 0.03 (0.23) | 0.161 |
| (g/Dl) | | HalfLytely ®** | 4.51 (0.26) | 4.50 (0.28) | −0.00 (0.22) | |
| Alk Phos | 40-135 | BLI-850 | 72.1 (20.9) | 73.0 (24.3) | 1.46 (10.9) | 0.716 |
| (U/L) | | HalfLytely ® | 72.6 (26.2) | 73.8 (29.5) | 1.96 (13.6) | |
| ALT | 0-47 | BLI-850 | 26.5 (19.1) | 27.7 (18.1) | 0.93 (8.8) | 0.422 |
| (U/L) | | HalfLytely ® | 26.9 (20.4) | 31.1 (52.2) | 4.20 (50.8) | |
| Amylase | 28-100 | BLI-850 | 68.5 (26.2) | 58.7 (22.3) | −11.1 (14.7) | 0.486 |
| (U/L) | | HalfLytely ® | 78.1 (93.0) | 62.2 (33.0) | −16.5 (97.3) | |
| Anion Gap | No Range | BLI-850 | 11.9 (2.05) | 12.4 (2.10) | 0.71 (2.70) | 0.551 |
| (mEq/L) | Available | HalfLytely ® | 12.1 (2.02) | 12.5 (2.29) | 0.53 (2.79) | |
| AST/SGOT | 0-37 | BLI-850 | 23.6 (8.9) | 26.1 (11.6) | 2.63 (6.5) | 0.994 |
| (U/L) | | HalfLytely ® | 24.0 (10.6) | 26.4 (17.5) | 2.61 (16.8) | |
| Bicarbonate | 20-31 | BLI-850 | 25.0 (2.5) | 24.3 (2.4) | −0.88 (2.6) | 0.200 |
| (mEq/L) | | HalfLytely ® | 24.9 (2.3) | 24.4 (2.3) | −0.51 (2.5) | |
| BUN | 9-24 | BLI-850 | 16.8 (5.1) | 13.3 (4.7) | −3.08 (3.6) | 0.222 |
| (mg/dL) | | HalfLytely ® | 16.7 (6.8) | 13.1 (6.0) | −3.59 (3.9) | |
| Calcium | 8.4-10.2 | BLI-850 | 9.83 (0.39) | 9.75 (0.41) | −0.08 (0.42) | 0.271 |
| (mg/dL) | | HalfLytely ® | 9.90 (0.45) | 9.76 (0.40) | −0.13 (0.43) | |
| Chloride | 95-113 | BLI-850 | 102.7 (2.6) | 103.3 (2.6) | 0.63 (2.4) | 0.177 |
| (mEq/L) | | HalfLytely ® | 102.7 (2.5) | 103.1 (2.4) | 0.27 (2.4) | |
| Creatine | F 24-170 | BLI-850 | 120.7 (111) | 152.1 (220) | 29.1 (198) | 0.192 |
| Kinase (U/L) | M 24-195 | HalfLytely ® | 116.2 (75) | 124.4 (124) | 6.2 (110) | |
| Creatinine | F 0.5-1.0 | BLI-850 | 0.97 (0.25) | 0.97 (0.24) | 0.00 (0.14) | 0.127 |
| (mg/dL) | M 0.6-1.4 | HalfLytely ® | 0.96 (0.35) | 0.99 (0.37) | 0.03 (0.12) | |
| Direct | 0.0-0.2 | BLI-850 | 0.12 (0.05) | 0.17 (0.06) | 0.05 (0.05) | 0.556 |
| Bilirubin | | HalfLytely ® | 0.12 (0.05) | 0.17 (0.08) | 0.05 (0.06) | |
| (mg/dL) | | | | | | |
| GFR | No Range | BLI-850 | 79.4 (19.5) | 79.5 (21.2) | −0.21 (14.6) | 0.072 |
| | Available | HalfLytely ® | 82.1 (20.0) | 79.3 (19.9) | −2.82 (11.5) | |
| GGT (U/L) | F 0-33 | BLI-850 | 36.4 (44.0) | 34.2 (35.4) | −1.38 (11.8) | 0.233 |
| | M 0-51 | HalfLytely ® | 34.0 (60.9) | 31.0 (30.4) | 0.73 (19.1) | |
| Glucose | 70-141 | BLI-850 | 105.1 (28) | 105.3 (31) | 0.82 (32) | 0.046 |
| (mg/dL) | | HalfLytely ® | 107.4 (32) | 100.4 (28) | −5.52 (25) | |
| Magnesium | 1.4-2.1 | BLI-850 | 1.76 (0.15) | 1.76 (0.15) | −0.01 (0.13) | 0.105 |
| (mEq/L) | | HalfLytely ® | 1.77 (0.15) | 1.74 (0.14) | −0.03 (0.14) | |
| Osmolality | 275-295 | BLI-850 | 288.5 (6.6) | 287.3 (5.6) | −0.88 (6.2) | 0.370 |
| | | HalfLytely ® | 288.1 (6.5) | 287.8 (6.5) | −0.18 (7.9) | |
| Phosphate | 2.4-4.9 | BLI-850 | 3.53 (0.59) | 3.45 (0.56) | −0.08 (0.65) | 0.390 |
| (mg/dL) | | HalfLytely ® | 3.59 (0.55) | 3.59 (0.55) | −0.02 (0.56) | |
| Potassium | 3.6-5.2 | BLI-850 | 4.31 (0.47) | 4.10 (0.39) | −0.19 (0.43) | 0.344 |
| (mEq/L) | | HalfLytely ® | 4.35 (0.46) | 4.12 (0.38) | −0.24 (0.46) | |
| Sodium | 134-146 | BLI-850 | 139.6 (2.4) | 140.0 (2.3) | 0.47 (2.2) | 0.459 |
| (mEq/L) | | HalfLytely ® | 139.7 (2.2) | 140.0 (2.1) | 0.29 (2.2) | |
| Total | 0.0-1.1 | BLI-850 | 0.44 (0.22) | 0.72 (0.32) | 0.28 (0.21) | 0.877 |
| Bilirubin | | HalfLytely ® | 0.44 (0.21) | 0.72 (0.35) | 0.27 (0.24) | |
| (mg/dL) | | | | | | |
| Total Protein | 6.1-7.9 | BLI-850 | 7.08 (0.37) | 7.15 (0.44) | 0.06 (0.38) | 0.182 |
| (g/dL) | | HalfLytely ® | 7.15 (0.44) | 7.14 (0.44) | 0.01 (0.36) | |
| Uric Acid | F 2.2-6.4 | BLI-850 | 5.50 (1.6) | 5.82 (1.5) | 0.39 (0.80) | 0.231 |
| (mg/dL) | M 3.1-8.8 | HalfLytely ® | 5.33 (1.4) | 5.82 (1.3) | 0.49 (0.77) | |

¹M = Male, F = Female;
²P-Value for treatment differences is from a one-way ANOVA
*Sulfate salt/PEG prep described in Example 1
**Bisacodyl/HalfLytely ® prep described in Example 1

Mean glucose values (mg/dL) rose 0.8 in the sulfate salt/PEG group versus a decrease of 5.5 in the bisacodyl/PEG group. Creatinine values for sulfate salt/PEG patients did not increase from baseline to Visit 2, while creatinine values for bisacodyl/PEG patients increased slightly (+0.03).

Patients in both treatment groups had BUN decreases and bilirubin increases after preparation which was not considered to be clinically significant.

### EQUIVALENTS

Those skilled in the art will recognize, or be able to ascertain, using no more than routine experimentation, numerous equivalents to the specific composition and procedures described herein. Such equivalents are considered to be within the scope of this invention, and are covered by the following claims.

1. A method for rapidly cleansing the small and large intestines and colon of a patient, comprising:
(a) orally administering an effective amount of an aqueous hypertonic sulfate solution to the patient to induce purgation, the solution not producing clinically significant electrolyte shifts, and not containing phosphates; and
(b) orally administering an effective amount of an aqueous isotonic solution containing an osmotic laxative to induce purgation,
the purgations induced by the solutions administered in (a) and (b) resulting in intestinal and colonic cleansing in less than about four hours from the administration of the sulfate solution.

2. The method of claim 1, wherein the aqueous, hypertonic solution comprises Na₂SO₄, K₂SO₄, and MgSO₄.

3. The method of claim 2, wherein the aqueous, hypertonic solution consist essentially of Na₂SO₄, K₂SO₄, and MgSO₄.

4. The method of claim 2, wherein the aqueous, hypertonic solution comprises about 8 g to about 26.5 g of Na₂SO₄, about 1.5 g to about 4.7 g of K₂SO₄, and/or about 1 g to about 2.4 g of MgSO₄.

5. The method of claim 3, wherein the aqueous, hypertonic solution consists essentially of about 8 g to about 26.5 g of Na₂SO₄, about 1.5 g to about 4.7 g of K₂SO₄, and/or about 1 g to about 2.4 g of MgSO₄.

6. The method of claim 1, wherein the aqueous, hypertonic solution contains about 72.9 mM to about 233 mM sulfate ion.

7. The method of claim 1, where the aqueous, hypertonic solution comprises about 155 mM sulfate ion.

8. The method of claim 1, wherein the aqueous isotonic solution comprises PEG.

9. The method of claim 8, wherein the isotonic solution is PEG 3350.

10. The method of claim 8, wherein the aqueous, isotonic solution comprises from about 105 g to about 315 g PEG.

11. The method of claim 10, wherein the aqueous, isotonic solution comprises about 118 g PEG.

12. The method of claim 10, wherein the aqueous isotonic solution comprises about 210 g PEG.

13. The method of claim 1, wherein the aqueous, isotonic solution further comprises Na₂SO₄, Na₂CO₃, NaCl, and KCl.

14. The method of claim 13, wherein the aqueous, isotonic solution further comprises about 5.0 g to about 18.0 g Na₂SO₄, about 1.7 g to about 5.5 g Na₂CO₃, about 1.5 g to about 4.4 g NaCl, and about 0.75 g to about 2.3 g KCl.

15. The method of claim 14, wherein the aqueous, isotonic solution further comprises about 11.37 g Na₂SO₄, about 3.37 g Na₂CO₃, about 2.93 g NaCl, and about 1.5 g KCl.

16. The method of claim 1, wherein the aqueous, isotonic solution further comprises Na₂CO₃, NaCl, and KCl.

17. The method of claim 16, wherein the aqueous, isotonic solution further comprises about 1.4 g to about 4.3 g Na₂CO₃, about 2.8 g to about 8.4 g NaCl, and about 0.4 g to about 1.1 g KCl.

18. The method of claim 17, wherein the aqueous, isotonic solution further comprises about 2.8 g Na₂CO₃, about 5.6 g NaCl, and about 0.7 g KCl.

19. The method of claim 2, wherein about 100 ml to about 900 ml of the aqueous, hypertonic sulfate solution is administered.

20. The method of claim 19, wherein about 100 ml to about 500 ml of the aqueous, hypertonic sulfate solution is administered.

21. The method of claim 19, wherein about 330 ml of the aqueous, hypertonic sulfate solution is administered.

22. The method of claim 19, wherein about 480 ml of the aqueous, hypertonic sulfate solution is administered.

23. The method of claim 8, wherein about 1 L to about 3 L of the aqueous isotonic solution is administered.

24. The method of claim 23, wherein about 2 L of the aqueous isotonic solution is administered.

25. The method of claim 1, wherein the aqueous, isotonic solution is administered immediately after ingestion of the aqueous, hypertonic sulfate solution.

26. The method of claim 1, wherein the aqueous, isotonic solution is administered about 0.5 hour to about 2 hours after ingestion of the aqueous, hypertonic sulfate solution.

27. The method of claim 1, wherein the water is orally administered after the step of orally administering the aqueous, hypertonic sulfate solution.

28. The method of claim 1, wherein the aqueous, isotonic solution is administered about 1 hour to about 2 hours after ingestion of the aqueous, hypertonic sulfate solution.

29. The method of claim 1, further comprising administering water immediately after administering the aqueous hypertonic solution.

30. A method for rapidly cleansing the small and large intestines and colon of a patient, comprising:
(a) orally administering about 100 ml to about 900 ml of an aqueous hypertonic solution consisting essentially of about 17.5 g Na₂SO₄, about 1.6 g MgSO₄, and about 3.1 g K₂SO₄ to induce purgation, the solution not producing any clinically significant electrolyte shifts, and not containing phosphates;
(b) orally administering water; and
(c) orally administering from about 1 L to about 3 L of an aqueous, isotonic solution comprising about 168 g PEG, about 11.37 g Na₂SO₄, about 3.37 g Na₂CO₃, about 2.93 g NaCl, and about 1.985 g KCl to induce purgation,
the purgations induced by the solutions administered in (a) and (b) resulting in intestinal and colonic cleansing in less than about four hours from the administration of the sulfate solution.

31. A method for rapidly cleansing the small and large intestines and colon of a patient, comprising:
(a) orally administering about 100 ml to about 900 ml of an aqueous hypertonic solution consisting essentially of about 17.5 g Na₂SO₄, about 1.6 g MgSO₄, and about 3.1 g K₂SO₄ to induce purgation, the solution not producing any clinically significant electrolyte shifts, and not containing phosphates;
(b) orally administering water; and
(c) orally administering from about 1 L to about 3 L of an aqueous, isotonic solution comprising about 210 g PEG, about 2.86 g Na₂CO₃, about 5.6 g NaCl, and about 0.74 g KCl to induce purgation,
the purgations induced by the solutions administered in (a) and (b) resulting in intestinal and colonic cleansing in less than about four hours from administration of the sulfate solution.
